(19) United States
(12) Patent Application Publication
(10) Pub. No.: US 2007/0225671 A1
(43) Pub. Date: Sep. 27, 2007

(54) DISPOSABLE SANITARY PERSONAL-CARE ARTICLE FOR ABSORBING BODILY MATERIAL

(76) Inventor: Bennet E. Angel JR., Sammamish, WA (US)

Correspondence Address: PERKINS COIE LLP;PATENT-SEA, P.O. BOX 1247, SEATTLE, WA, 98111-1247, US

(21) Appl. No.: 11/757,208

(22) Filed: Jun. 1, 2007

Related U.S. Application Data
(63) Continuation-in-part of application No. 11/454,681, filed on Jun. 15, 2006, now abandoned.
(60) Provisional application No. 60/691,209, filed on Jun. 15, 2005.

(51) Int. Cl. A61F 13/15

(52) U.S. Cl. 604/385.17; 604/385.101; 604/385.01

(57) ABSTRACT

The present invention is directed to a disposable sanitary article for use with a woman's body in the area of the pudendum. The sanitary article of one embodiment comprises a pad portion having a fluid-permeable surface portion and an absorbent material adjacent to the surface portion. The pad portion is configured to be positioned exterior of the pudendum and to absorb a bodily fluid from the pudendum. A reservoir member is coupled to the pad portion and that defines a reservoir adjacent to the absorbent material. The reservoir member is shaped and sized to fit on a portion of the pudendum. The reservoir member is configured to receive and contain the bodily fluid therein until the bodily fluid can be substantially absorbed by the absorbent material.

### CROSS-REFERENCE TO RELATED APPLICATION(S)

This application is a continuation-in-part patent application that claims priority to U.S. patent application Ser. No. 11/454,681, entitled DISPOSABLE SANITARY PERSONAL-CARE ARTICLE FOR ABSORBING BODILY MATERIAL, filed Jun. 15, 2006, which is a non-provisional patent application that claims priority to U.S. Provisional Patent Application No. 60/691,209, entitled A RAISED MEMBER FOR THE COLLECTION AND DISTRIBUTION OF MENSTRUAL MATERIAL FOR TAMPONS AND SANITARY PADS, filed Jun. 15, 2005, both of which are incorporated herein in their entirety by reference thereto.

### TECHNICAL FIELD

The present invention is related to disposable absorbent articles and more particularly to sanitary personal-care articles for women.

### BACKGROUND

Conventional sanitary pad and tampon designs are configured to collect and retain menstrual material and to protect clothing from the material. The conventional designs, however, have drawbacks that can lead to leakage of menstrual material onto the wearer's undergarments or other clothing. A conventional sanitary pad or tampon is considered to have failed if menstrual material is not caught by the intended absorption device when used in accordance with the manufacturer's directions. Often these design failures become more pronounced when there is a rapid or large expulsion of fluid and/or more viscous or solid material, collectively referred to as menstrual material.

Sanitary pads are designed to sit in a wearer's clothing and absorb the flow of menstrual material onto the absorbent material as it is expelled from the woman's body. Conventional sanitary pads, however, often do not stay in place and can move around in the clothing and relative to the woman's pudendum during use. This movement causes the risk of possible leakages onto the wearer's clothing if the pad is not correctly positioned to catch the menstrual material. If there is a rapid or large expulsion of menstrual material, as often happens, the pad material often cannot absorb all the menstrual material fast enough, thereby causing the pad design to fail. To compensate for these design shortcomings, manufacturers typically make pads much larger with more absorbent material needed to hold the amount of menstrual material collected before being discarded. In other words, only the center of the pad will actually be used before it is discarded, leaving the pad's ends and sides unutilized.

Larger pad sizes also cause a further problem regarding reduced comfort in wear. Typically, the larger the pad, the more uncomfortable it is to wear. Larger pads are often more visible within clothing, which is also typically not desired. The larger pads also often deform into an inverted U or V shape when worn. The wider the pad, the greater chance that the pad will deform into an inverted U or V shape. This inverted shape is not conducive for the catching of liquids; quite the opposite. The deformation turns the pad's shape into that of a roof-shape that allows menstrual material to travel down the pad before it can be absorbed, thereby contributing to pad failure.

Conventional tampons also have design problems. Menstrual material can often bypass a tampon within the vaginal canal for a variety of reasons, even when used per manufacturer instructions. Even simple body movements, when menstrual material is being expelled, can cause tampon failure. Another problem with conventional tampons is the fact that the user cannot accurately tell when the tampon is sufficiently filled and ready to be removed. If the tampon is removed too soon, the tampon will be partially dry, and dry fibers that make up the tampon can be left inside the vaginal canal. The tampon fibers left behind can cause undesirable dryness within the vaginal canal. It is also undesirable to leave these fibers in the vaginal canal for a number of health reasons. If, on the other hand, the tampon is allowed to completely fill, which is preferred for removal, the tampon may fail because it cannot absorb any more menstrual material. As a result, the excess menstrual material can bypass the tampon, resulting in failure of the device. Accordingly, there is a need for an improved disposable personal-care article useable to collect and absorb menstrual material and other bodily fluid.

### SUMMARY

The present invention is directed to a disposable sanitary article that overcomes drawbacks experienced in the prior art and provides other benefits. One embodiment of the invention provides a disposable sanitary article for use by a woman. The sanitary article comprises a pad portion having a fluid-permeable surface portion and an absorbent material adjacent to the surface portion. The pad portion is configured to be positioned exterior of the woman's pudendum and to absorb a bodily material from the pudendum. The absorbent material has a top portion configured to face the pudendum and a bottom portion opposite the top portion. A rigid reservoir member is coupled to the absorbent material so the rigid reservoir member will be intermediate the absorbent material and the pudendum. The rigid reservoir member defines a reservoir with a fixed reservoir shape adjacent to the top portion of the absorbent material during use by the woman. The reservoir member is shaped and sized to fit on and press against a portion of the pudendum and to maintain its fixed reservoir shape during use by the woman to receive and contain the bodily material therein until the bodily material can be substantially absorbed by the absorbent material. The reservoir member is configured to provide structural rigidity to the absorbent material adjacent to the reservoir member to hold the reservoir open and to prevent the absorbent material from collapsing, bending, or substantially deforming relative to the pudendum during use by the woman.

Another embodiment of the invention provides a disposable sanitary article for collecting bodily material. The sanitary article comprises a pad member having a fluid-permeable surface portion and an absorbent material adjacent to the surface portion and configured to absorb bodily material passing through the surface portion. A rigid reservoir member is attached to the surface portion of the pad member and projects away from the surface portion. The reservoir member defines a reservoir adjacent to the surface portion and configured to contain the bodily material therein until the bodily material can be substantially absorbed by the absorbent material. The reservoir member provides structural rigidity to the absorbent material adjacent to the reservoir to hold the reservoir in a fixed open condition and to substantially prevent the absorbent material adjacent to the reservoir from collapsing, bending, or substantially deforming.

Another embodiment of the invention provides a disposable sanitary article for use with a woman's body in the area of the pudendum. The sanitary article comprises an absorbent pad having a fluid absorbent material therein. The pad portion is configured to be positioned exterior of the pudendum and to absorb menstrual material from the pudendum. The pad has a substantially rigid reservoir portion positioned about a reservoir adjacent to the absorbent material. The reservoir is shaped and sized to fit on an external portion of the pudendum in alignment with the vaginal opening therein and to receive the menstrual material from the pudendum until the menstrual material can be substantially absorbed by the absorbent material. The reservoir member provides structural rigidity to the absorbent material adjacent to the reservoir to hold the reservoir in a fixed open condition and to substantially prevent the absorbent material adjacent to the reservoir from collapsing, bending, or substantially deforming. The sanitary article further comprises a tampon having a body portion and a flexible engagement member. The pad is connected to the engagement member and is movable along the engagement member relative to the tampon.

The foregoing and other aspects of the invention will now be described in more detail with reference to the accompanying drawings. This Summary section is provided to introduce in a simplified manner aspects and features further described below in the Detailed Description section and illustrated in the figures. This Summary section is not intended to limit the scope of the claimed subject matter.

### BRIEF DESCRIPTION OF THE DRAWINGS

FIG. 1 is a top isometric view of a sanitary pad device in accordance with one embodiment of the present invention, wherein the device has an absorbent pad with a padded reservoir member located on the pad.

FIG. 2 is an enlarged cross-sectional view taken substantially along line 2-2 of FIG. 1 showing a portion of the reservoir member, with a liquid-proof padding thereon.

FIG. 3 is a cross-sectional view taken substantially along line 3-3 of FIG. 1.

FIG. 4 is a cross-sectional view of a portion of an absorbent pad and a reservoir member having padding covering a portion of the reservoir member in accordance with another embodiment.

FIG. 5 is a top plan view of a sanitary pad device in accordance with another embodiment of the present invention.

FIG. 6 is a side elevation view of the sanitary pad device of FIG. 5.

FIG. 7 is a top isometric view of the sanitary pad device of FIG. 5.

FIG. 8 is a top plan view of a sanitary pad device with a reservoir member padded with a portion of the same material that makes up the pad itself. The padding is shown adjacent to the reservoir member prior to being folded over the reservoir member for purposes of illustration.

FIG. 9 is a cross-sectional view taken substantially along lines 9-9 of FIG. 8, wherein the padding on the left side is shown folded over the reservoir member, and the padding is shown on the right side not yet folded over the reservoir member for purposes of illustration.

FIG. 10 is an enlarged cross-sectional view of the reservoir member of FIG. 9 with padding material applied thereto in accordance with another embodiment, wherein the padding has a liquid-proof covering or coating and is folded over the reservoir member.

FIG. 11 is a top plan view of a sanitary pad device of another embodiment shown with a thin fluid-permeable fabric covering a portion of the pad around the reservoir member.

FIG. 12 is a side elevation view of the sanitary pad device of FIG. 11.

FIG. 13 is a top isometric view of the sanitary pad device of FIG. 11.

FIG. 14 is a cross-sectional view of a sanitary pad device in accordance with another embodiment, wherein a portion of a reservoir member is shown covered by a padded portion, and another portion of the reservoir member is shown not yet covered by a padded portion for purposes of illustration.

FIG. 15 is a cross-sectional view of a sanitary pad device in accordance with another embodiment.

FIG. 16 is a cross-sectional view of a sanitary pad device in accordance with yet another embodiment.

FIG. 17 is a top plan view of a sanitary pad device with several concentric reservoir members in accordance with another embodiment.

FIG. 18 is a top plan view of a sanitary pad device with a reservoir member having multiple reservoirs in accordance with another embodiment.

FIG. 19 is a top plan view of a sanitary pad device with a reservoir member having multiple reservoirs in accordance with yet another embodiment.

FIG. 20 is a top plan view of a sanitary pad device with a reservoir member having multiple reservoirs in accordance with yet another embodiment.

FIG. 21 is a top plan view of a sanitary pad device with a plurality of reservoir members positioned to form a reservoir in accordance with another embodiment.

FIG. 22 is a side elevation view of a sanitary device with a tampon shown with an applicator in accordance with another embodiment.

FIG. 23 is a side elevation view of the sanitary pad device with the tampon of FIG. 22, wherein the applicator is shown spaced apart from an absorbent body portion of the tampon prior to the applicator being removed from the sanitary device.

FIG. 24 is an isometric view of the sanitary pad device of FIG. 23 with the applicator removed from the tampon.

FIG. 25 is a top plan view of a tampon applicator with a longitudinal slit along two adjoining pieces of the applicator in accordance with another embodiment.

FIG. 26 is a cross-sectional view of the two pieces of the tampon applicator of FIG. 25.

FIG. 27 is an isometric view of the tampon applicator of FIG. 25.

FIG. 28 is an isometric view of a sanitary pad device in accordance with another embodiment of the invention.

FIG. 29 is a cross-sectional view of the sanitary pad device taken substantially along lines 29-29 of FIG. 28.

FIG. 30 is a top plan view of a base layer of absorbent material and a reservoir portion of the sanitary pad device of FIG. 28, and with a top layer of absorbent material not shown for purposes of clarity.

FIG. 31 is a top plan view of a base layer of absorbent material and a reservoir portion of the sanitary pad device of FIG. 28 in accordance with another embodiment.

FIG. 32 is a top plan view of an absorbent top layer of the sanitary pad device of FIG. 28, shown removed from the base layer.

FIG. 33 is a top plan view of an absorbent top layer of the sanitary pad device of FIG. 28 in accordance with another embodiment.

FIG. 34 is a partially cut-away top isometric view of a sanitary pad device in accordance with another embodiment, wherein the reservoir portion is shown partially cut away for purposes of illustration.

FIG. 35 is an enlarged cross-sectional view taken substantially along line 35-35 of FIG. 34.

FIG. 36 is an isometric view of a sanitary pad device in accordance with another embodiment of the invention.

FIG. 37 is a side elevation view of the sanitary pad device of FIG. 36.

FIG. 38 is a top plan view of the sanitary pad device of FIG. 36 shown with a top layer removed for purposes of illustration.

### DETAILED DESCRIPTION

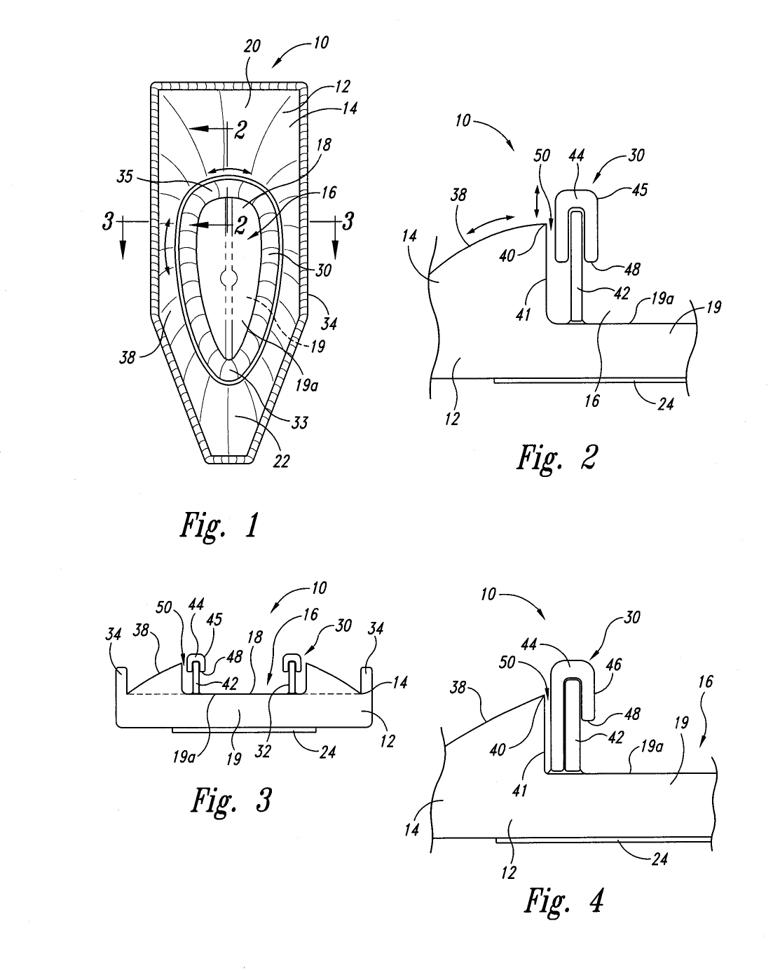

Embodiments of the present invention include disposable sanitary personal-care articles, including sanitary articles for use with a woman's body in the area of the pudendum to receive and contain bodily fluid, including menstrual material. Several specific details of the invention are set forth in the following detailed description and in FIGS. 1-38 to provide a thorough understanding of embodiments of the invention. One skilled in the art, however, will understand that the present invention may have additional embodiments, and that other embodiments of the invention may be practiced without one or more of the specific features described below. In other instances, well-known structures, material, or operations are not shown or described in order to avoid obscuring aspects of the invention.

As discussed above, sanitary pads in the prior art have experienced the problem of menstrual material flowing off the side of the pad. The present inventor recognized that the prior art, in its attempt to provide a comfortable product, created a flexible pad that is highly susceptible to deformation, bending, or folding in a convex shape relative to the wearer's pudendum. As a result, the expelled menstrual material will flow over the pad and leak past the edges of the pad before the pad can absorb the menstrual material.

Some prior art devices have attempted to incorporate flexible barrier walls to form a large or even small enclosure to receive the menstrual material depending on the design. The present inventor discovered that this version of the prior art still experienced failures notwithstanding the flexible barrier walls because the flexible pad will still invert and fold. This inversion of the flexible pad when the pad is being worn severely limits or obviates the effectiveness of any enclosure because the walls when folded tip downward on the sides of the pad, so the menstrual materials will flow over the sides. The present inventor further recognized that if the pad and the barrier walls that form an enclosure are flexible, then the “space” within the enclosure is not protected. Accordingly, when the sanitary pad folds, bend, or otherwise creases, the absorbent material on the inside of the enclosure will protrude into the “space,” thereby reducing or eliminating the intended space inside the enclosure, so the “space” effectively ceases to exist because the pad is folded and each side is tipped downwardly.

The disposable sanitary personal care articles of embodiments of the present invention include the sanitary pad device having an absorption material and a reservoir positionable against an external portion of a female wearer's pudendum area. The reservoir is configured to receive, control, and contain menstrual material or other bodily material expelled from the pudendum adjacent to the absorbent pad until the material can be absorbed by the pad. The reservoir can be formed by a substantially rigid reservoir member attached to a pad containing one or more absorption materials.

The reservoir member is shaped, sized, and configured to support the pad so the pad under the reservoir is held open and does not excessively bend, fold, crease, or deform during use. As a result of the substantially rigid reservoir member providing the structural support to the pad to maintain the integrity of the pad's portion below the reservoir, the absorbent pad can be substantially smaller than conventional pads and extend radially past the periphery of the rigid reservoir member while maintaining exceptional control and absorption of the flow of menstrual material when in use. In another embodiment, the absorbent pad's periphery is substantially the same size and shape as the periphery of the reservoir member's periphery. The absorption material in the pad can include conventional hydrophilic material capable of absorbing menstrual blood and other bodily fluids and material. Such absorption material can be a fiber material (e.g., cellulose fiber, synthetic fiber, wood pulp, etc.), absorbent gelling material, or other conventional absorption material used in sanitary pad material, tampon material, diaper material, or other material used to absorb bodily fluids.

The reservoir member fits comfortably over and engages an exterior portion of the wearer's pudendum (i.e., the vulva, including the labia majora and minora, and the vaginal opening) to collect and hold the discharged fluids until the absorption material can absorb them. In at least one embodiment, the reservoir member fits with a gasket-like engagement against a portion of the labia majora and in alignment with vaginal opening. In one embodiment, the reservoir member is shaped and sized so a posterior end is positioned generally between the vaginal opening and the anus. The anterior area of the reservoir member is positioned on the labia majora below an area generally between the vaginal opening and the clitoris. In one embodiment, the substantially rigid reservoir member is approximately 1.5-inches long and approximately 1-inch wide. In other embodiments, however, the reservoir member can be larger or smaller to fit over more or less of the pudendum while maintaining alignment with the vaginal opening. In other embodiments, the reservoir member can have a uniform height, or it can have different heights along portions of the reservoir member to generally correspond to the external shape or contours of the pudendum.

The reservoir member can be made of material including, but not limited to, plastic, metal, wire, cardboard, cotton, rubber, or other sufficiently stiff material that will substantially maintain the shape of the reservoir while the sanitary pad device is in use. The reservoir member can be liquid-proof, or can be covered with a liquid-proof covering or coating. The reservoir member may be padded. There may also be multiple reservoir members working in conjunction with one another to provide one or more reservoirs that will collect and hold menstrual material or other bodily material until they are absorbed by the absorption material while maintaining the structural stability of the pad and the integrity of the reservoir. In one embodiment, the reservoir is retained in a fixed open position by a brace, such as one or more cross brace members, spanning across the reservoir against or immediately adjacent to the pad. The brace provides structural rigidity to the sanitary pad device to help the pad maintain its shape adjacent to the reservoir.

One embodiment of the rigid reservoir member of the sanitary pad device has a teardrop shape that defines a rigid teardrop reservoir that sits above absorbent material. The reservoir member is designed to fit comfortably within the confines of the area of the woman's body in which it sits, namely in the existing space between the legs, and engagement with the pudendum (as discussed above) and just anterior of the woman's buttocks, such that these confines of the woman's body keep the reservoir and pad properly placed.

The teardrop-shaped reservoir member has a narrow posterior end, and the sides of the reservoir member diverge from the narrow end of the teardrop to the wider anterior end. This shape follows the natural V shape of the vaginal/pudenda area, from the narrow area located between the legs, just rearward of the vaginal opening, widening out at the area adjacent to the labia majora and below the lower abdomen. So, the reservoir member acts like a wedge, with the narrow end resting comfortably against the beginning of the buttocks, and the sides widening out, confined by the inner thighs. The buttocks are used as the stop that ensures the reservoir member (and thus, the pad) cannot move backward and out of alignment with the vaginal opening. The reservoir member is shaped so the inner thighs ensure that the reservoir member cannot move side to side, and the wearer's underwear or other garment ensures that the pad and reservoir member cannot slip forward out of alignment with the vaginal opening. Thus, the reservoir member is “boxed” in by the buttocks at the posterior end, the legs for the sides, the vaginal area being the top, and the underwear acting as the anterior end and bottom. Accordingly, the reservoir member is “boxed-in”, so it stays positioned under the vaginal opening to accept menstrual material when the sanitary pad is in use. This “boxed-in” effect is maintained at least in part because the sanitary pad device is correctly sized and shaped. In addition, the reservoir always remains open under the vaginal opening because the reservoir member is substantially rigid, such that the pad and reservoir will not collapse, fold, or otherwise deform so as to compromise the integrity of the reservoir. Also, it is important to note that the entire area of the reservoir remains open on or immediately adjacent to the vaginal opening and the reservoir remains in its position.

The inventor recognized that the wearer's legs may move relative to the side of the reservoir, and the buttocks may have some movement relative to the reservoir at the very beginning of the crease, but the area of the pudendum on which the reservoir member sits has substantially no movement, even when the rest of the body is in motion. Accordingly, the inventor recognized that a properly sized and shaped rigid reservoir member can be used to fit so well into the area and keep such good contact with the pudendal region, that it stays positioned correctly while maintaining comfort for the wearer.

The reservoir member, along with being properly sized and shaped, is substantially rigid so it stays open during use to protect the “space” inside the reservoir from being compromised. The rigid reservoir member will not bend or fold along the horizontal plane during normal use. So, when the rigid reservoir member is properly adhered to the top of the absorbent material, the absorbent material under the reservoir will not bend, fold, or substantially deform, so the “space” inside the reservoir will always be maintained independent of normal body motion by the wearer.

If the reservoir member were not sufficiently rigid, the reservoir member and pad could bend or fold, for example, along a longitudinal crease line, causing the absorbent material below the reservoir to be thrust upward into the reservoir, thereby compromising the integrity of the reservoir. The inventor recognized that this action could cause at least a couple of problems. First, if the reservoir was folded at the narrow posterior end and the absorbent material was pushed up into the reservoir near that posterior end, the relative height of the wall defining the reservoir at that area would be much shorter and less capable of containing a normal to heavy flow of menstrual material. Accordingly, this section of the reservoir would provide an easy spot for the menstrual material to flow over that section of the wall. Second, if the reservoir were to crease down the longitudinal crease line and the reservoir walls were now on both sides of the crease, the reservoir would effectively cease to exist. This would mean that the reservoir walls would now simply act like barrier walls for slowing menstrual materials from spilling off the sides of the pad.

The inventor also recognized that the properly sized, shaped, and positioned rigid reservoir acts as a conduit of menstrual material, providing total control of the material from the time it is expelled from the vaginal opening to the time it is fully absorbed by the absorbent material. Since the reservoir, which fully maintains its integrity during normal movement by the wearer, is gently pressed against the soft folds of the wearer's skin, and it is adhered to the absorbent material, the reservoir acts like a “pipe” (although it be a very short pipe) between the two, controlling the flow of materials until they can be fully absorbed.

As long as the “conduit,” or rigid reservoir member, stays positioned properly and the “space” inside the reservoir and the integrity of the absorbent material below the reservoir are maintained, portions of the absorbent material outside the reservoir can deform and conform to each individual wearer's body, thereby increasing comfort with no loss to performance. Since it is the reservoir that controls the flow of menstrual material from the vaginal opening to the absorbent material inside the reservoir, the absorbent body can be made extremely narrow in the crotch region; it just needs to be as wide as the reservoir member. This means that sanitary pads suitable to control up to an extra heavy menstrual flow can be small and narrow, while providing a more comfortable pad.

Menstrual material consists of solids, semisolids, and liquid. The solid and semisolid menstrual material often just rests on top of a pad until it dries, causing an unpleasant wet feeling for the wearer. The space between the wearer's pudendum and the top of the absorbent material, provided by the walls of the rigid reservoir, keeps the solid and semisolid material away from the user's skin, providing a drier feeling than otherwise possible.

FIG. 1 is a top isometric view of a disposable sanitary pad device 10 in accordance with an embodiment of the present invention. The illustrated sanitary pad device includes an absorbent pad 12 having a peripheral edge portion 14 and a substantially rigid reservoir member 30 positioned generally centrally on an upper surface 18 of the pad. The reservoir member defines a reservoir 16 adjacent to a portion of the pad's upper surface. The pad's upper surface faces toward the wearer's pudendum when in use, and a bottom side of the pad faces away from the pudendum (and typically toward an undergarment worn by the wearer). In one embodiment, the pad has a thin, fluid impermeable liquid barrier on its bottom side. The pad contains an absorption material 19 configured to absorb and retain the menstrual material. The pad is sized to contain enough absorption material to fully absorb up to an extra heavy flow of menstrual material. As discussed in greater detail below, the sanitary pad device is configured so that the pad does not need a significant amount of extra absorption material as is common in conventional sanitary pads.

The pad 12 is shaped to fit comfortably between the wearer's legs against the wearer's pudendum and adjacent to the wearer's buttocks. In the illustrated embodiment, the pad has a generally rectangular anterior portion 20, and a posterior portion 22 tapers rearwardly to a narrowed posterior end. The pad is shaped and sized so the anterior portion is positioned generally against a portion of the labia majora below the area generally between the urethra and the mons veneris, and preferably just rearward of the clitoris. The pad is shaped and sized so the narrowed posterior end will be positioned approximately at or against the beginning of the wearer's buttocks. The narrow end is configured to gently engage the area near the beginning of the buttocks, so that the pad will not slip rearwardly in use. Instead the beginning of the wearer's buttocks will help position the pad and the reservoir member in proper position relative to the vaginal opening. Because the narrowed end starts near the beginning of the user buttocks and does not extend farther back, the pad remains closely contoured to the wearer's body. Accordingly, the pad is substantially undetectable when looking at the wearer. Also, since the pad is so narrow, small and contoured to the wearer's body, the pad sits very comfortably between the wearer's legs without the pad moving around and bunching up within the wearer's underwear or other undergarments.

As best seen in FIG. 3, the sanitary pad device 10 of the illustrated embodiment has an adhesive material 24 on the underside of the pad. The adhesive material is configured to releasably secure the pad 12 to a wearer's undergarment or other clothing. The adhesive can be a traditional adhesive backing for sanitary pads. The device can include a release liner on the adhesive material to protect the adhesive until the wearer is ready to attach the device to her undergarment or other clothing.

The sanitary pad device 10 has a substantially rigid reservoir member 30 that defines a reservoir 16 adjacent to an upper surface 18 of the pad 12 and adjacent to the absorption material 19. In the illustrated embodiment, the reservoir member 30 is a ring attached to the top of the pad to form a raised structure with sidewalls 32 that define the sides of the reservoir 16. The reservoir member is shaped to be generally teardrop-shaped with the posterior end 33 being narrower than the anterior end 35 (FIG. 1), so the reservoir member will comfortably fit against and around an external portion of the pudendum in a gasket-like engagement. Accordingly, a portion of the pudendum is within the reservoir, so the engagement between the reservoir member and the pudendum will help hold the sanitary device in proper position on the wearer's body.

The reservoir member 30 of the illustrated embodiment is positioned on top of, and attached to, the absorption material 19 or to a fluid-permeable liner 19a covering the absorption material. The reservoir member is configured to be placed over the pudendum so the reservoir will receive, collect, and hold the menstrual material until the material can be absorbed by the absorption material. The reservoir member will hold the menstrual material within the reservoir 16 and provide the absorption material as much time as needed to fully absorb the discharge. Because the sanitary pad device 10 is positioned around an exterior portion of the pudendum, the reservoir is generally aligned with the vaginal canal, so all the discharged material will be caught in the reservoir until absorbed. The rigid reservoir member maintains the shape and integrity of the reservoir and the absorbent material below the reservoir, so there is very little possibility of device failure.

In the illustrated embodiment, the peripheral edge portion 14 of the pad 12 has an outer wall portion 34 projecting away from the pad's upper surface 18. In the event that the sanitary pad device 10 is dislodged relative to the wearer's body and some menstrual material engages the pad outside the reservoir 16, the outer wall portion will help contain the menstrual material on the pad until absorbed by the absorption material, rather than leaking off the pad and onto the wearer's clothing. The outer wall portion 34 of the illustrated embodiment is integrally formed in the peripheral edge portion of the pad. Other embodiments can have an outer wall portion formed by a raised member adhered or otherwise attached around the pad.

FIG. 2 is an enlarged cross-sectional view taken substantially along line 2-2 of FIG. 1 showing the reservoir member 30 and absorption material 19. FIG. 3 is a cross-sectional view taken substantially along line 3-3 of FIG. 1 showing the pad 12, the reservoir member 30, and the reservoir 16. The pad of the illustrated embodiment has sloped intermediate portions 38 that slope upwardly from the perimeter edge portion 14 toward the reservoir member. The sloped intermediate portion has an inner edge 40 and an inner sidewall 41 adjacent to the reservoir member. The inner sidewall is configured to help support the reservoir member around the reservoir to maintain a comfortable and close fit against the external portions of the pudendum.

In another embodiment, the rigid reservoir member 30 can be an integral portion of the inner sidewall 41 around the reservoir 16 and configured to receive and closely fit to the external portion of the pudendum as discussed above. In the illustrated embodiment, the rigid reservoir member can have a slight outward curve that provides a small amount of lateral flex radially inwardly when pushed from the sides. The flexibility of the reservoir member selected during manufacturing can be based upon the material used, the thickness of the material used, and the reservoir member's structural design. The reservoir member, however, does not flex or collapse under normal pressure applied vertically. Accordingly, the rigid reservoir member helps the entire sanitary pad device 10 maintain its shape, so the pad will not bunch up, collapse, or fold when it is being worn.

The reservoir member 30 may be made of a material or combination of materials substantially impervious to fluids, such as plastics, metals, wire, rubber, or other suitable materials. In another embodiment, the rigid reservoir member can be made of one or more materials, such as compressed cotton, cardboard, or other suitable material, that may absorb some fluids retained in the reservoir 16 without losing the rigidity and structural integrity of the reservoir member. In another embodiment, the reservoir member is made of an absorbent material having a fluid-impervious coating thereon that prevents or slows down absorption of the fluid by the reservoir member. In the illustrated embodiment, the reservoir member has a body portion 42 securely connected to the pad's upper surface 18 to provide structural stability to the pad so the reservoir 16 will not collapse under normal use. The body portion can be adhered to the upper surfaces as to provide the structural rigidity, with some flexibility as discussed above. In other embodiments, the body portion can be partially embedded into the pad. In another embodiment, the body portion can be integrally formed in the pad.

The reservoir member 30 of the illustrated embodiment has soft padding 44 attached to the top of the body portion 42. The soft padding is configured to engage the wearer's pudendum to provide a comfortable fit against the wearer's body. The soft padding of the illustrated embodiment is covered with a liquid-proof covering or coating 46. The soft padding can be adhered directly to the body portion by glue, tape, or other connection method. The soft padding is configured to form an inner lip 48 on the inside of the reservoir member. In another embodiment, the inner lip can be integrally formed on the body portion 42. The inner lip allows menstrual material to flow down into the reservoir 16 and into the pad for absorption, and the inner lip also helps prevent backflow of the menstrual material out of the reservoir, such as when the wearer is lying down and the device is tipped away from a generally horizontal orientation.

In one embodiment, the reservoir member 30 is formed and assembled with the padding 44 separately, then attached to the pad 12 via glue, tape, or other common method of adhesion. The reservoir member can be formed by a single member, or by a plurality of members. The reservoir member as illustrated is a single closed, teardrop-shaped ring. In another embodiment, the reservoir member can have different shapes and can be configured as an open structure that contains and controls the menstrual material before it is absorbed. For example, in one embodiment, the reservoir member is configured to form a reservoir 16 that has a tortuous path along which menstrual material may move until it can be absorbed by the absorption material in the pad portion.

In the illustrated embodiment, the pad 12 includes the sloped intermediate portion 38, and the reservoir member 30 is spaced radially inward from the inner sidewall 41, so there is a small space 50 between the inner sidewall and the reservoir member 30. This space allows the pad's sloped intermediate portion to slightly compress under pressure, while the rigid reservoir member remains in contact with the wearer's body.

FIG. 4 is an enlarged cross-sectional view of a portion of the pad 12 and reservoir member 30 having a liquid-proof padding 44 covering the body portion 42 in accordance with another embodiment. A portion of the padding extends over the top of the body portion and forms the inner lip 48 along inside of the reservoir member. Another portion of the padding extends down along the outer side of the body portion and is securely attached to the pad. The padding is also adhered to the reservoir member's body portion, which helps anchor the reservoir member to the pad while providing a comfortable, padded interface with the wearer's body.

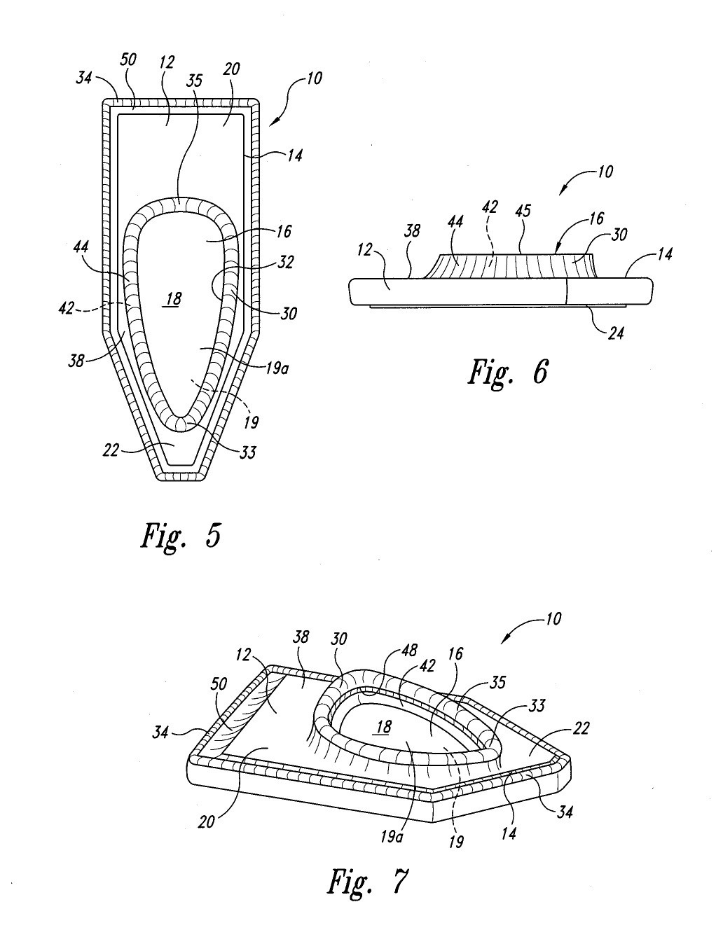

FIG. 5 is a top plan view of a sanitary pad device 10 in accordance with another embodiment. FIG. 6 is a side elevation view of the sanitary pad device of FIG. 5, and FIG. 7 is a top isometric view of the sanitary pad device of FIG. 5. In this embodiment, the upper surface 18 of the pad 12 is substantially flat, such that the pad's intermediate portion 38 has a substantially uniform thickness. The reservoir member 30 is adhered or otherwise securely attached to the pad, as discussed above. The reservoir member can have the same construction as discussed above. In another embodiment, the reservoir member can be a ring, such as a continuous band or O-ring attached to the pad and configured to define the reservoir 16. The reservoir member can have a continuous or non-continuous teardrop shape, oval shape, or other shape. The ring can also have a layer of padding to add to the comfort of the wearer.

In the illustrated embodiment, the pad 12 has a trough 52 formed in the peripheral edge portion 14 radially outward from the reservoir member. The trough of the illustrated embodiment is a recessed trough formed in the pad. Accordingly, the trough does not add to the thickness of the pad. The trough provides an extra protection to help prevent any stray menstrual material not captured in the reservoir 16 from flowing over the side of the pad.

In another embodiment, the peripheral edge portion 14 of the pad 12 has a short, raised wall projecting up from the pad's upper surface and forming a barrier to block any stray menstrual fluid not captured in the reservoir 16 from flowing over the edge of the pad. In another embodiment, the short, raised wall can be provided adjacent to the trough 52 in the pad as discussed above. The short, raised wall can be an integral portion of the pad, or the wall can be a structure adhered to the pad portion. The short, raised wall around the peripheral edge portion 14 can be configured to add some structural stability to the pad to help the pad maintain its shape when worn.

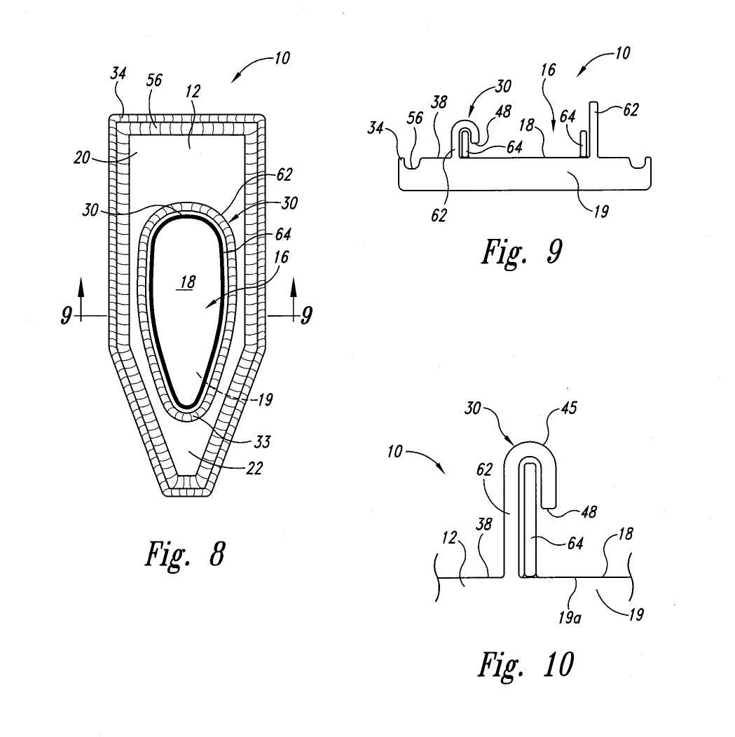

FIG. 8 is a top plan view of another embodiment of a sanitary pad device 10 with a padded reservoir member 30. FIG. 9 is a cross-sectional view taken substantially along line 9-9 of FIG. 8, and FIG. 10 is an enlarged cross-sectional view of the reservoir member of FIG. 8. In the illustrated embodiment, the reservoir member has padding 62 on a structural body 64. The padding is integrally connected to the pad 12 and is formed by the same absorption material in the pad. The padding can be coated or covered by a fluid-impervious material. In another embodiment, the padding (or some of the padding) can be configured to absorb fluids. The padding of the illustrated embodiment is shown in FIG. 10 and in FIG. 9 (on the left side of the drawing) in an assembled configuration wrapped over and attached to the structural body. The padding is shown on the right side of FIG. 9 adjacent to the structural body prior to being wrapped over and secured in place to the structural body. Accordingly, the absorption material forms the padding to provide a comfortable fit for the wearer.

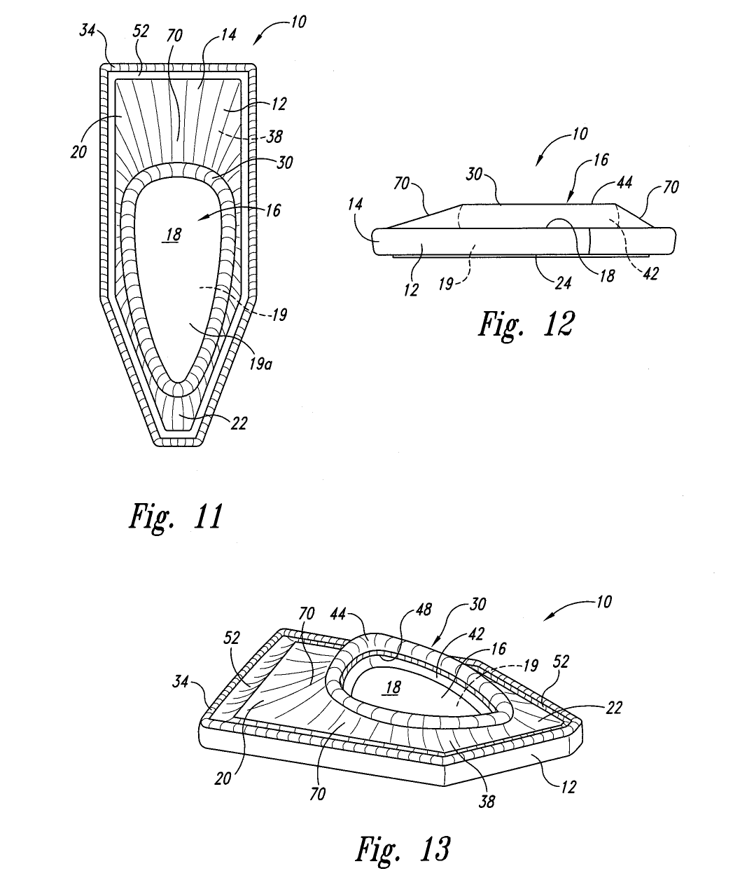

FIG. 11 is a top plan view of a sanitary pad device 10 in accordance with another embodiment. FIG. 12 is a side elevation view of the sanitary pad device of FIG. 11, and FIG. 13 is an isometric view of the sanitary pad device of FIG. 11. In this embodiment, the sanitary pad device 10 has a similar construction as discussed above in connection with FIG. 5. The sanitary pad device also has an elastic membrane 70 that extends from the peripheral edge portion 14 of the pad to the top of the reservoir member 30. The membrane can be fluid-permeable, so menstrual material can pass through the membrane and into the absorption material 19 in the pad. The membrane stretches over the pad's intermediate portion 38 so as to help resist or prevent the pad portion from folding into an inverted V- or U-shape. In the illustrated embodiment, the membrane spans between the peripheral edge portion and the reservoir member, so there is nothing between the membrane and the top of the pad. When the sanitary pad device is gently pushed against the pudendum via the wearer's clothing, the membrane can be pushed down and touch the absorbent material, thereby holding the pad in a shape generally contoured to the wearer's body.

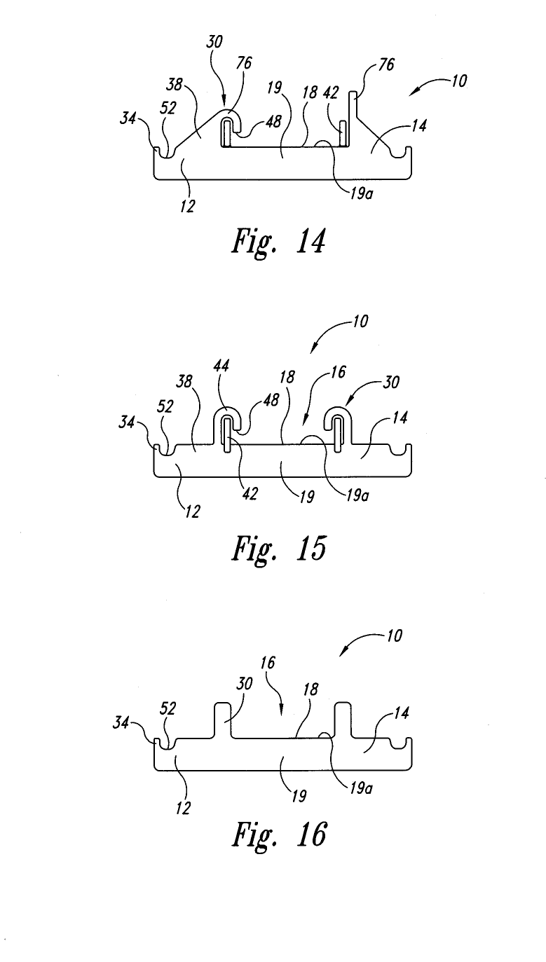

FIG. 14 is a cross-sectional view of a sanitary pad device 10 in accordance with another embodiment. The pad 12 has a sloped intermediate portion 38 integrally connected to flexible interior flanges 76 configured to form a padding portion for the reservoir member 30. The flexible flanges wrap over and securely connect to the body portion 42 of the reservoir member. In another embodiment, the flexible flanges can be separate pieces of material adhered to the sloped intermediate portion of the pad and to the body portion of the reservoir member. The peripheral edge portion 14 of the pad can have a trough 52 therein as discussed above. In another embodiment, the peripheral portion of the pad has a flat configuration without the trough.

FIG. 15 is a cross-sectional view of the sanitary pad device 10 in accordance with another embodiment. In the illustrated embodiment, the pad 12 of the device has a substantially uniform thickness, and the rigid reservoir member 30 extends upwardly from the pad's upper surface. The body portion 42 of the reservoir member of the illustrated embodiment is adhered to the top of the pad portion. The padding 44 is shown embedded at one end into the pad adjacent to the reservoir member. The other end of the padding wraps over the top of the body portion and is adhered in place to provide the padded reservoir member that comfortably engages the wearer about the pudendum.

FIG. 16 is a cross-sectional view of the sanitary pad device 10 in accordance with another embodiment. In this embodiment, the reservoir member 30 is a raised structure integrally connected to the pad and formed from the absorption material 19 of the pad. The absorption material forming the reservoir member may be tightly compressed so it would only absorb a minute amount of fluid before the absorption material in the pad would fully absorb the menstrual material. In one embodiment the reservoir member is made from the tightly compressed absorption material so as to maintain its stiffness and rigidity. The reservoir member is covered with a liquid-proof covering or coating to restrict or limit how much fluid is absorbed by the compressed absorption material.

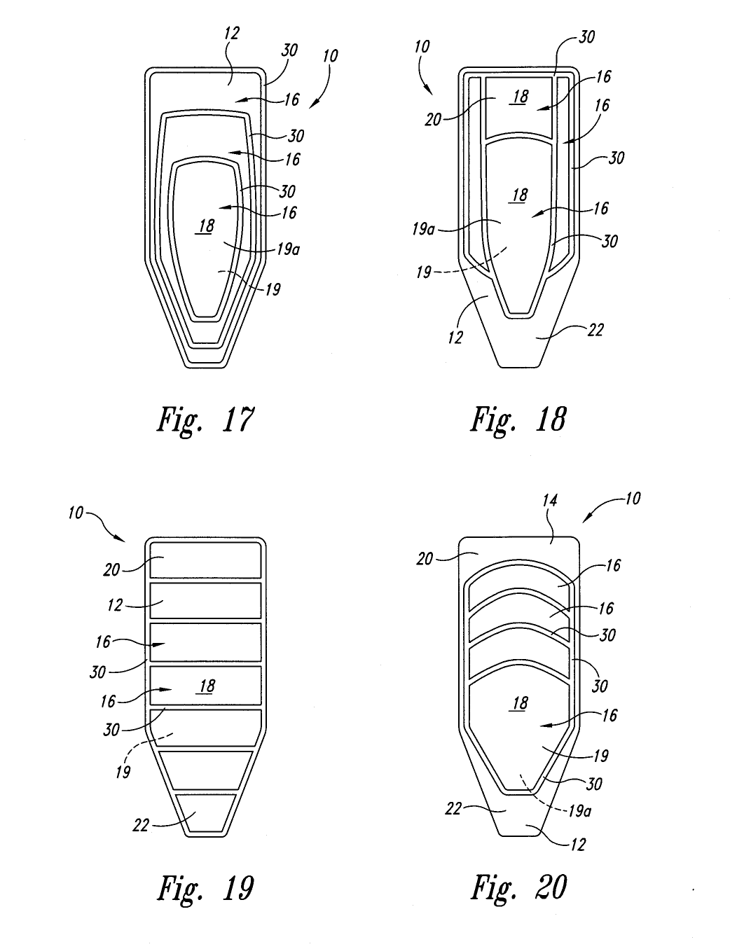

FIGS. 17, 18, 19 and 20 show top plan views of sanitary pad devices 10 in accordance with alternate embodiments. In the illustrated embodiments, the rigid reservoir members 30 are connected to the pad 12, and configured to define a plurality of reservoirs 16 adjacent to the absorption material 19 in the pad 12. In addition, the sanitary pad device can have a plurality of reservoir members that work in conjunction with one another to form the multiple reservoirs. For example, the device of FIG. 17 has a plurality of generally concentric reservoir members attached to the pad to form the multiple reservoirs. The reservoir members can have the same height relative to the upper surface 18 of the pad, or in other embodiments, they may have different heights. As seen in FIGS. 18, 19, and 20, the reservoir members can form a plurality of adjacent reservoirs having a selected shape and orientation relative to the pad. For example, reservoirs can be formed adjacent to each other and sized based upon expected or potential leakage from one reservoir to another reservoir during use.

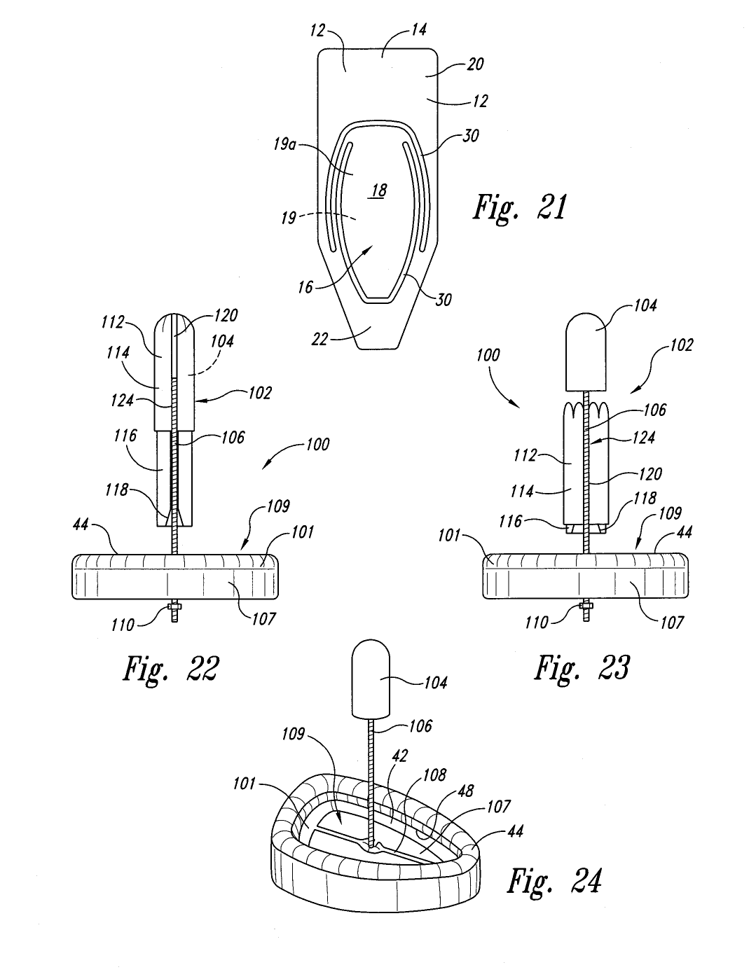

FIG. 21 is a top plan view of a sanitary pad device 10 in accordance with another embodiment. This embodiment shows that the reservoir member 30 does not have to be a fully closed area to achieve the same function as a closed reservoir member. For example, the reservoir member can have multiple pieces attached to the pad portion adjacent to each other without forming a fully closed reservoir. The reservoir member and the resulting reservoir, however, are configured to receive, contain, and direct the menstrual material relative to the pad portion so as to give the absorption material enough time to absorb the menstrual material before it flows out of the reservoir toward the peripheral edge portion of the pad.

In another embodiment, the sanitary pad device 100 is used in conjunction with a tampon 102. The sanitary pad device has substantially the same construction as described above, except as discussed below. FIG. 22 is a side elevation view of the sanitary pad device 100 connected to the tampon 102 in an applicator 112. FIG. 23 is a side elevation view of the sanitary pad device 100 connected to the tampon, with the applicator 112 in the compressed condition after the tampon has been deployed. FIG. 24 is an isometric view of the sanitary pad device and tampon assembly in accordance with one embodiment.

The tampon 102 has an absorbent body portion 104 connected to a string 106. The string is connected to the sanitary pad device 100. In one embodiment, the string extends into the reservoir 109 and through the pad 107, so the pad can be slid closer or farther away from the tampon as desired or needed by the wearer. The string has a knot 110 or other retention device that prevents the string from pulling out of the pad portion. In another embodiment, a retainer device 108 is connected to the pad portion and to the string, so the string and tampon will remain connected to the sanitary pad device. The retention device of one embodiment is a brace spanning across the reservoir against or immediately adjacent to the pad. The brace can provide some structural rigidity to the sanitary pad device to help the pad maintain its shape. The retention device can also be moveably connected to the tampon's string. Accordingly, the sanitary pad device can slide along the string to adjust the distance between the tampon's body portion and the pad.

The tampon 102 is configured to be inserted into the vagina to absorb and collect menstrual material in a conventional way. The sanitary pad device 100 is configured to be positioned against the exterior portion of the wearer's pudendum, as discussed above. The reservoir 109 in the sanitary pad device will catch any menstrual material that may bypass the tampon. This allows the user to let the tampon fill completely, and the user can check to see whether the tampon is in fact full by looking at the pad rather than by removing the tampon. This also avoids premature removal of tampons, which could leave the dry tampon fibers in the vagina and cause vaginal dryness.

In use, the tampon 102 is inserted into the vagina, dispensed via an applicator 112 (discussed in greater detail below), and then the sanitary pad device 100 is slid up the tampon's string until the reservoir member 101 engages the pudendum. The string will hold the sanitary pad device in position snugly against the pudendum with the reservoir 109 below the vaginal opening. Since the sanitary pad device will be snugly held in place at least in part by the tampon and its string, the size of the pad in the sanitary pad device can be minimized. In one example, the shape of the pad portion is approximately the same shape as the reservoir member. Thus, the device is extremely small, comfortable, efficient, and virtually invisible within the wearer's clothing. The sanitary pad device 100 can have an adhesive on the bottom of the pad 107 to releasably attach to the wearer's clothing. Other embodiments do not need the adhesive material to comfortably retain the pad in place against the wearer's pudendum because the sanitary pad device works with the tampon to hold the sanitary pad device in place against the wearer.

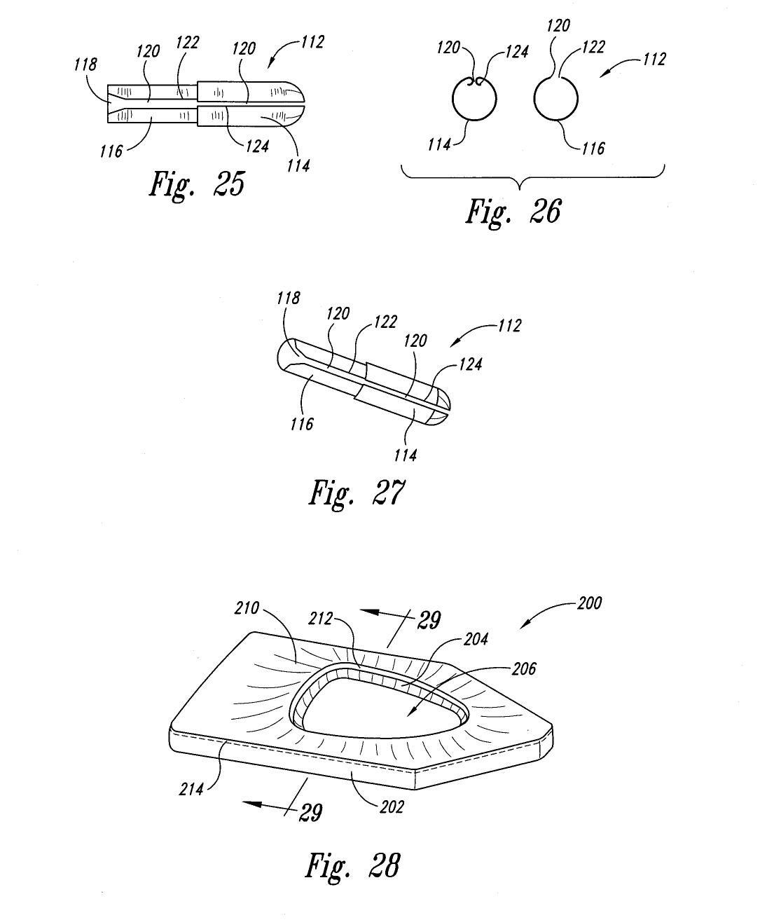

FIG. 25 is a top plan view of the tampon applicator 112 used to insert the tampon 102, which is attached to the sanitary pad device 100. The illustrated applicator 112 has two pieces: a top piece 114 and a bottom piece 116. Each of the top and bottom pieces has a longitudinal slit 120 extending along its length. The slit is configured to allow the tampon string to pass therethrough so the applicator can be removed from the tampon and the sanitary pad device. In the illustrated embodiment, the bottom piece of the applicator has a V-shaped notch 118 therein that enables the string to be easily moved into and through the slot during removal.

FIG. 26 is a cross-sectional view of the top and bottom pieces 114 and 116 of the applicator 112 separated from each other. FIG. 27 is an isometric view of the applicator with both pieces of the applicator joined together. The top piece 114 of the applicator, which houses the tampon (not shown), has edges 122 that define the longitudinal slit 120, and these edges are folded over to create an alignment guide 124. The alignment guide slideably receives the edges of the applicator's bottom piece 116 that define the longitudinal slit therein. Accordingly, the bottom piece of the applicator will slide along the alignment groove of the top piece as the tampon is pushed out the end of the applicator top piece. The alignment guide also helps keep the longitudinal slit in both applicator pieces aligned. So, after the tampon has been deployed into the vagina, the longitudinal slits in the applicator will remain aligned so the string can easily pass therethrough as the applicator is being removed from the string.

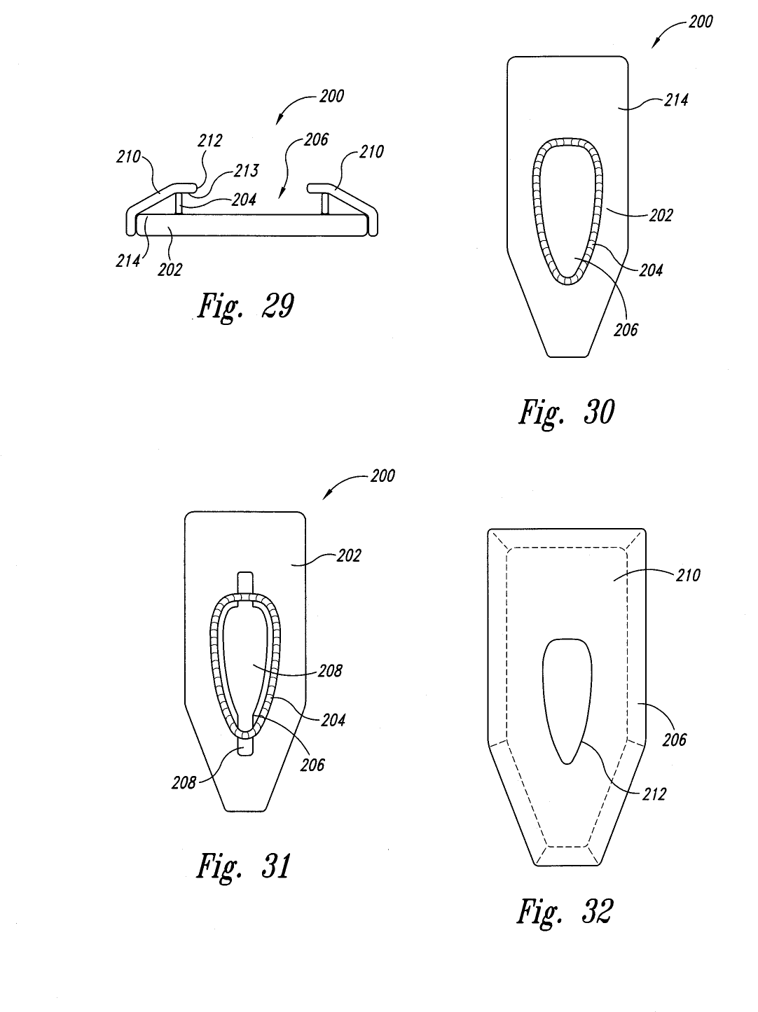

FIG. 28 is an isometric view of a sanitary pad device 200 in accordance with another embodiment of the invention. FIG. 29 is a cross-sectional view of the sanitary pad device taken substantially along lines 29-29 of FIG. 28. FIG. 30 is a top plan view of a base layer of absorbent material and a reservoir member of the sanitary pad device of FIG. 28 with a top layer of absorbent material not shown for purposes of clarity. The sanitary pad device 200 has an absorbent base pad portion 202 and the reservoir member 204 that defines a reservoir 206, substantially as described above. The base pad portion in the illustrated embodiment is a substantially planar member, and the reservoir member is adhered or otherwise attached to the top surface of the base pad portion.

In another embodiment shown in FIG. 31, the base pad portion 202 can have a channel or other recess 208 formed therein and positioned below the reservoir member. The recess creates a deeper reservoir or pocket that can receive and contain the menstrual material. The recess can have a shape generally corresponding to the shape of the reservoir member. Alternatively, the recess can extend past the periphery of the reservoir member, as shown in FIG. 31. The reservoir member 204 is attached to the base pad portion so as to provide structural support that helps prevent or resist the base pad portion from folding, collapsing, or otherwise closing the recess during use.

Referring back to FIG. 29, the illustrated sanitary pad device includes an absorbent top layer 210 coupled to the base layer and positioned over the reservoir member 204. The top layer also has an aperture 212 formed therein and shaped to substantially correspond to the shape of the reservoir. The top layer is positioned on the base pad portion so that the aperture is positioned over the reservoir 206. Accordingly, the top layer does not cover the reservoir. The top layer, however, does provide padding over the reservoir member, so that additional padding on the reservoir member is not needed.

In the illustrated embodiment, the portion of the top layer 210 defining the aperture 212 can be configured to form a lip 213 around the top of the reservoir 206 to help prevent backflow of the menstrual material from the reservoir, as discussed above. Accordingly, the top layer will not interfere with the menstrual material flowing into the reservoir. The top layer is also configured to catch and absorb any menstrual fluid that may miss the reservoir. The absorbent top layer of the illustrated embodiment is made of the same material used for the base pad portion. The top layer can be made of other materials in other embodiments.

FIG. 32 is a top plan view of an absorbent top layer 210 of the sanitary pad device 200 of FIG. 28. The top layer of the illustrated embodiment has a peripheral shape and size generally corresponding to the shape and size of the base pad portion 202 (FIG. 29). The top layer has a peripheral edge portion 214 that wraps at least partially around the edges of the base pad portion 202. In this embodiment, the periphery edge portion of the top layer can be adhered or otherwise connected to the top and/or sides of the base pad portion (fully or partially) around the peripheral edge portion 214. In one embodiment, the top layer is glued to the base pad portion, although other attachment mechanisms can be used. The top layer can also be adhered to the reservoir member 204 to retain the top layer aperture in alignment with the reservoir. In another embodiment, the top layer is not adhered to the reservoir member, thereby allowing for some relative movement between the two components.

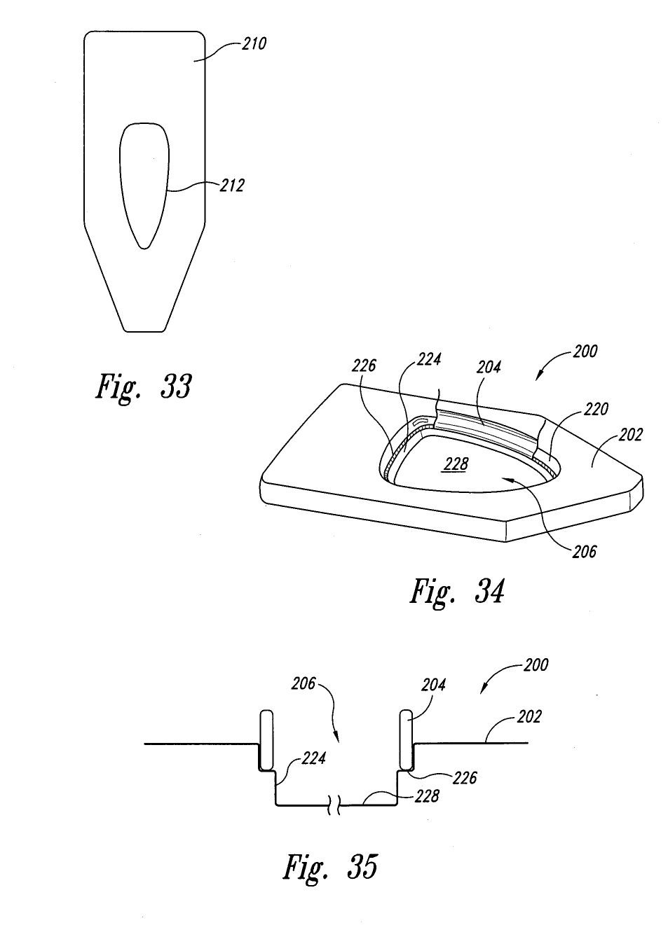

FIG. 33 is a top plan view of the top layer 210 of the sanitary pad device 200 shown removed from the base layer of an embodiment. In this embodiment, the top layer is similar to the top layer discussed above, but it is sized so its peripheral edge portions 214 are above the edges of the base pad portion 202. In this embodiment, the periphery of the top layer can be adhered or otherwise connected to the top surface of the base pad portion (fully or partially) around the peripheral edge portion 214.

FIG. 34 is a partially cut-away top isometric view of a sanitary pad device 200 in accordance with another embodiment, wherein the rigid reservoir member 204 is shown partially cut away for purposes of illustration. FIG. 35 is an enlarged cross-sectional view taken substantially along line 35-35 of FIG. 34. In this embodiment, the absorbent base pad portion 202 has a recess 220 formed therein that receives the reservoir member and forms the reservoir 206. Accordingly, the menstrual material will collect in the reservoir and be absorbed through the bottom of the recess into the absorbent material in the base pad portion. In the illustrated embodiment, the walls 224 defining the recess in the base pad portion have a stepped configuration providing a shelf 226 that supports the reservoir member above the base 228 of the recess. The recess and the shelf can be configured so the reservoir member is raised above the surface of the base pad portion. The reservoir member provides structural support to the base pad portion to help maintain the shape of the reservoir when the sanitary device pad is being worn.

In another embodiment, the recess 220 in the base pad portion 202 can be configured with or without a step and so that the rigid reservoir member 204 is received therein. The top edge of the reservoir member can be substantially flush with the top surface of the base pad portion 202. The reservoir member and the reservoir 206 defined thereby work in the same manner as discussed above to receive and contain the menstrual material until it can be absorbed by the absorbent material. The rigid reservoir member also provides structural stability to the base pad portion to help keep it from collapsing or folding during use as discussed above.

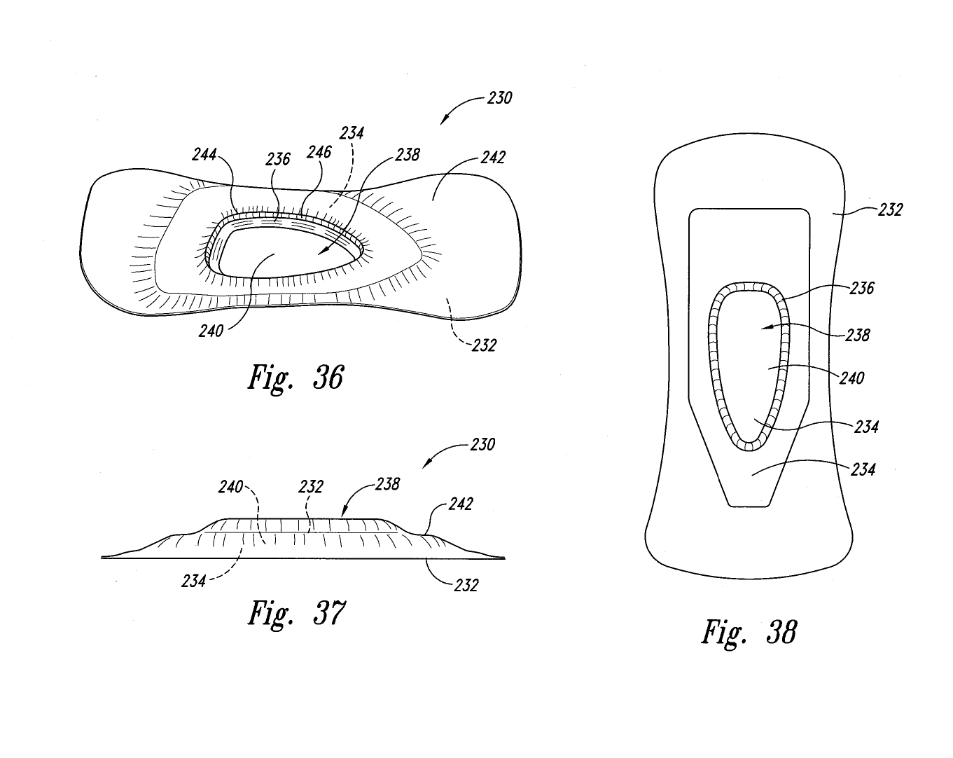

FIG. 36 is an isometric view of a sanitary pad device 230 in accordance with another embodiment of the invention. FIG. 37 is a side elevation view of the sanitary pad device of FIG. 35. FIG. 38 is a top plan view of the sanitary pad device of FIG. 36 shown with a top layer removed for purposes of illustration. The sanitary pad device of the illustrated embodiment has a base sheet 232 made of a flexible material having a shape that generally corresponds to the shape of a conventional sanitary pad or panty liner (with or without wings). The base sheet is a substantially fluid-impervious material, although other materials can be used. In one embodiment, the base sheet is a thin sheet of plastic material, although other materials can be used. A layer of tape or other adhesive can be attached to the outer surface of the base sheet. The adhesive is configured to releasably attach the sanitary pad device to the wearer's undergarment or other clothing.

The sanitary pad device 230 of the illustrated embodiment has an absorbent base pad portion 234 attached to the base sheet 232. A reservoir member 236 defining the reservoir 238 is coupled to the base pad. The reservoir member and reservoir are adjacent to the base pad and configured so the reservoir will receive and contain the menstrual material therein until it can be absorbed by the absorbent material 240 in the base pad, substantially as discussed above. The absorbent base pad is attached to a generally central portion of the base sheet. The absorbent base pad is substantially smaller than the base sheet while providing all of the absorption capabilities needed to handle virtually any menstrual flow, from light to very heavy. The base sheet is very flexible, such as a thin plastic material, so that the base sheet does not provide discomfort to the wearer. The base pad and reservoir member (with or without the padding discussed above) are small and fit comfortably against the wearer's pudendum.

As best seen in FIGS. 36 and 37, the sanitary pad device 230 of the illustrated embodiment has a top sheet 242 attached to the base sheet 232 and extends over the base pad portion 234 and the reservoir member 236. The top sheet is a thin flexible sheet with a shape that generally corresponds to the shape of the base sheet, although other shapes can be used. The top sheet has an aperture 244 formed therein and positioned over the reservoir, so the top sheet does not cover the reservoir. The top sheet can be made of an absorbent material, so that the top sheet can provide padding over the reservoir member. Accordingly, additional padding on the reservoir member may not be needed.

The shape of the aperture 244 substantially corresponds to the shape of the reservoir 238. The portion of the top sheet 242 defining the aperture can be configured to form a lip 246 over the reservoir to help prevent backflow of the menstrual material from the reservoir. Accordingly, the top sheet will not interfere with the menstrual material flowing into the reservoir. The top sheet, however, is configured to catch and absorb any menstrual fluid that may miss the reservoir.

In one embodiment, the absorbent top sheet 242 of the illustrated embodiment is made of the same material used for the base pad portion 234. The top sheet can be made of other materials in other embodiments. The top sheet, however, is thin and flexible enough to provide no discomfort to the wearer. The sanitary pad device, however, will appear to have substantially the same shape and size as a conventional sanitary pad or panty liner, thereby providing emotional comfort to a wearer who may not want to try new things. The sanitary pad device, however, is substantially more comfortable and more effective than conventional sanitary pad products.

From the foregoing, it will be appreciated that specific embodiments of the invention have been described herein for purposes of illustration, but that various modifications may be made without deviating from the spirit and scope of the invention. Accordingly, the invention is not limited except as by the appended claims.

I claim:

1. A disposable sanitary article for use with a woman's body in the area of the pudendum, comprising:
a pad portion having a fluid-permeable surface portion and an absorbent material adjacent to the surface portion and configured to be positioned exterior of the pudendum and to absorb a bodily material from the pudendum, the absorbent material having a top portion configured to face the pudendum; and
a rigid reservoir member coupled to the absorbent material so the rigid reservoir member will be intermediate the absorbent material and the pudendum, the rigid reservoir member defines a reservoir with a fixed reservoir shape adjacent to the top portion of the absorbent material during use by the woman, the reservoir member being shaped and sized to fit on and press against a portion of the pudendum, the reservoir member being shaped and sized to maintain its fixed reservoir shape to receive and contain the bodily material therein until the bodily material can be substantially absorbed by the absorbent material, the reservoir member providing structural rigidity to the absorbent material adjacent to the reservoir member to hold the reservoir open and to prevent the absorbent material in communication with the reservoir from collapsing, bending, or substantially deforming relative to the pudendum during use by the woman.

2. The disposable sanitary article of claim 1 wherein the reservoir member is a ring fixed around its perimeter to the absorbent material.

3. The disposable sanitary article of claim 1 wherein the reservoir member includes a rigid ring having a continuous non-circular shape with a first end portion and a second end portion narrower than the first end, the ring being positioned on the pad portion so the second end is posterior from the first end when the sanitary article is worn by the woman.

4. The disposable article of claim 3 wherein the ring has a substantially teardrop shape.

5. The disposable sanitary article of claim 1 wherein the reservoir member is a non-continuous member with a plurality of rigid segments positioned to define the reservoir.

6. The disposable sanitary article of claim 1, the woman having a buttocks portion posterior of the pudendum, and wherein the reservoir member has an anterior end and a posterior end narrower than the anterior end, the posterior end being shaped and sized to fit under the pudendum and forwardly adjacent to the buttocks portion whereby the buttocks portion blocks the reservoir member from moving rearwardly relative to the pudendum when in use by the woman.

7. The disposable sanitary article of claim 1, the woman having a perineum portion posterior of the pudendum and anterior of a buttocks portion, and wherein the reservoir member has an anterior end and a diverging posterior end narrower than the anterior end, the posterior end being shaped and sized to fit under the pudendum and terminating immediately adjacent to the perineum portion forward of the buttocks portion.

8. The disposable sanitary article of claim 1 wherein the pad portion has a perimeter portion and the reservoir member is positioned immediately adjacent to the entire perimeter portion.

9. The disposable sanitary article of claim 8 wherein the reservoir member is shaped and sized to fit within a periphery of the pudendum when in use by the woman.

10. The disposable sanitary device of claim 1, further comprising a tampon having an absorbent body portion and a flexible engagement member attached to the body portion, the flexible engagement member being slideably connected to the pad portion.

11. The disposable sanitary device of claim 10 wherein the flexible engagement member is a string that extends through the pad portion, the pad portion being slideable along the string relative to the tampon.

12. The disposable sanitary device of claim 10 wherein the flexible engagement member is a string that extends through the pad portion and through the reservoir, the pad portion being slideable along the string relative to the tampon.

13. The disposable sanitary article of claim 1, further comprising a fluid-permeable cover layer attached to the pad member and covering the reservoir member.

14. The disposable sanitary article of claim 1 wherein the pad portion has a peripheral portion having a collection channel therein in communication with the absorbent material.

15. The disposable sanitary article of claim 1 wherein the pad portion has a peripheral portion and a perimeter wall projecting away from the peripheral portion.

16. The disposable sanitary device of claim 1 wherein the reservoir member is made of a non-absorbent material.

17. The disposable sanitary device of claim 1 wherein the pad portion is configured to be positioned in a wearer's undergarment, and further comprising a retention portion coupled to the pad portion and being releasably connectable to the wearer's undergarment to retain the reservoir member about the pudendum.

18. A disposable sanitary article for collecting bodily material, comprising:
a pad member having a fluid-permeable surface portion and an absorbent material adjacent to the surface portion and configured to absorb bodily material passing through the surface portion; and
a substantially rigid reservoir member attached to the surface portion of the pad member and projecting away from the surface portion, the reservoir member extending about a reservoir adjacent to the surface portion and configured to contain the bodily material therein until the bodily material can be substantially absorbed by the absorbent material, the reservoir member providing structural rigidity to the absorbent material adjacent to the reservoir to hold the reservoir in a fixed open condition and to substantially prevent the absorbent material adjacent to the reservoir from collapsing, bending, or substantially deforming.

19. The disposable sanitary article of claim 18 wherein the reservoir member is a ring fixed around its perimeter to the absorbent material.

20. The disposable sanitary article of claim 18 wherein the reservoir member includes a rigid ring having a non-circular shape with a first end portion and a second end portion narrower than the first end portion, the ring being positioned on the pad member so the second end is posterior from the first end.

21. The disposable sanitary article of claim 18 wherein the reservoir member has an anterior end and a posterior end narrower than the anterior end, the posterior end being shaped and sized to restrict posterior movement of the reservoir member relative to a wearer when in use.

22. The disposable sanitary article of claim 18 wherein the reservoir member has an anterior end and a diverging posterior end narrower than the anterior end, the posterior end being shaped and sized to terminate immediately adjacent to a buttocks portion of a woman when the sanitary article is in use.

23. The disposable sanitary article of claim 18 wherein the pad member has a perimeter portion and the reservoir member is positioned immediately adjacent to the entire perimeter portion.

24. The disposable sanitary article of claim 18 wherein the reservoir member is shaped in a substantially rigid, closed ring.

25. The disposable sanitary device of claim 18, further comprising a tampon having an absorbent body portion and a flexible engagement member attached to the body portion, the flexible engagement member being slideably connected to the pad member.

26. The disposable sanitary article of claim 18, further comprising a fluid-permeable cover layer attached to the pad portion and positioned to cover the surface portion and the reservoir member.

27. The disposable sanitary article of claim 18 wherein the pad portion has a peripheral portion having a collection channel therein in communication with the absorbent material.

28. The disposable sanitary article of claim 18 wherein the reservoir member is a non-continuous member with a plurality of rigid segments positioned to define the reservoir.

29. The disposable sanitary device of claim 18 wherein the pad member is configured to be positioned in a wearer's undergarment, and further comprising a retention portion coupled to the pad portion and being releasably connectable to the wearer's undergarment to retain the reservoir member about the external genitalia.

30. A disposable sanitary article for use with a woman's body in the area of the pudendum, comprising:
an absorbent pad assembly having a pad portion with a fluid absorbent material therein, the pad portion being configured to be positioned exterior of the pudendum and to absorb menstrual material from the pudendum, the pad assembly having a substantially rigid reservoir member and a reservoir adjacent to the absorbent material, the reservoir member being shaped and sized to fit on a portion of the pudendum, the reservoir member configured to receive and contain the menstrual material therein until the menstrual material can be substantially absorbed by the absorbent material, the reservoir member providing structural rigidity to the absorbent material adjacent to the reservoir to hold the reservoir in a fixed open condition and to substantially prevent the absorbent material adjacent to the reservoir from collapsing, bending, or substantially deforming; and
a tampon having a body portion and a flexible engagement member, the pad assembly being connected to the engagement member and being movable along the engagement member relative to the tampon.

31. The disposable sanitary article of claim 30 wherein the reservoir member has a perimeter and is sealably fixed around its perimeter to the absorbent material.

32. The disposable sanitary article of claim 30 wherein the reservoir member has an anterior end and a posterior end narrower than the anterior end, the posterior end being shaped and sized to engage a portion of the woman's body and be blocked from rearward movement of the reservoir member relative to the woman's body when in use.

33. The disposable sanitary article of claim 30 wherein the reservoir member has an anterior end and a diverging posterior end narrower than the anterior end, the posterior end being shaped and sized to terminate fit immediately adjacent to a buttocks portion of a woman when the sanitary article is in use.

34. The disposable sanitary article of claim 30 wherein the pad member has a perimeter portion and the reservoir member is positioned immediately adjacent to the entire perimeter portion.

35. The disposable sanitary article of claim 30 wherein the flexible engagement member is a string and the pad is slideable along the string relative to the tampon.

36. The disposable sanitary article of claim 30 wherein the reservoir member includes a rigid ring projecting away from a surface of the pad portion and defining the reservoir adjacent to the absorbent material.

37. The disposable sanitary device of claim 30 wherein the pad assembly is configured to be positioned in a wearer's undergarment, and further comprising a retention portion coupled to the pad portion and being releasably connectable to the wearer's undergarment to help retain the reservoir member about the pudendum.
